(19) United States
(12) Patent Application Publication
(10) Pub. No.: US 2021/0401407 A1
(43) Pub. Date: Dec. 30, 2021

(54) IDENTIFYING AN INTERVNTIONAL DEVICE IN MEDICAL IMAGES

(71) Applicant: KONINKLIJKE PHILIPS N.V., EINDHOVEN (NL)

(72) Inventors: Hongxu YANG, EINDHOVEN (NL); Alexander Franciscus KOLEN, EINDHOVEN (NL); Caifeng SHAN, VELDHOVEN (NL); Peter Hendrik Nelis DE WITH, SON EN BREUGEL (NL)

(21) Appl. No.: 17/290,792

(22) PCT Filed: Oct. 31, 2019
(86) PCT No.: PCT/EP2019/079878

§ 371 Date: May 3, 2021

Related U.S. Application Data
(60) Provisional application No. 62/754,250, filed on Nov. 1, 2018.
(60) Provisional application No. 62/909,392, filed on Oct. 2, 2019.

(51) Int. Cl. A61B 8/08; A61B 8/00; G06N 3/08

(52) U.S. Cl. CPC A61B 8/5207; G06N 3/08; A61B 8/461; A61B 8/0841

(57) ABSTRACT

Images may be preprocessed to select pixels or voxels of interest prior to being analyzed by a neural network. Only the pixels or voxels of interest may be analyzed by the neural network to identify an object of interest. One or more slices may be extracted from the voxels of interest and provided to the neural network for analysis. The object may be further localized after identification by the neural network. The preprocessing, analysis by the neural network, and/or localization may utilize pre-existing knowledge of the object to be identified.

### RELATED APPLICATIONS

This application claims priority to U.S. Provisional Application No. 62/754,250 filed on Nov. 1, 2018, and U.S. Provisional Application No. 62/909,392 filed on Oct. 2, 2019, the contents of which are incorporated by references herein for any purpose.

### TECHNICAL FIELD

The present disclosure pertains to imaging systems and methods for identifying an object in images. In particular, imaging systems and methods for identifying an interventional device in medical images.

### BACKGROUND

Clinicians rely on medical images before, during, and after interventional procedures. Medical images provide insight into the underlying tissue below the skin surface, and also allow the clinician to see foreign objects within the body. During an interventional procedure, medical images can be of particular usefulness in allowing a clinician to see the locale of a medical device (such as a catheter, guidewire, implant) being used in the procedure. The usefulness, however, depends on the accuracy in which the medical device can be detected within the image—as sometimes the location of the medical device may not be readily apparent in noisy or lower quality medical images. The detection of devices within images may be automated using one of many image processing techniques at varying degrees of success.

Additionally, some imaging modalities, like x-ray, require radiation, contrast fluids which can add to procedure length and inhibit both visual and automated image detection. Ultrasound is an attractive alternative to x-ray imaging, as it is radiation-free and provides flexibility with 2D (plane), 3D (volumetric) and 4D (volumetric and time) image datasets. Despite these advantages, images generated from ultrasound are often of low resolution and low contrast the 3D space, making it difficult for clinicians to timely localize a medical device in a procedure.

### SUMMARY

The present disclosure describes systems and methods for enhancing the detection of medical devices or other objects in images and shorten the computational time to detect the devices in the images, enabling real-time applications. This may improve clinical results and reduce procedure time. In particular, the systems and methods may enable object detection (e.g. catheter, guidewire, implant) using techniques that focus object detection on candidate pixels/voxels within an image dataset. The image dataset may include a two-dimensional (2D), three-dimensional (3D), or four-dimensional (4D) dataset. In some embodiments, a preset model based on the object may be used to detect the candidate pixels/voxels based on image data correlated to the object. The preset model may be supplied by the system or selected by the user. The preset model may include one or more filters, algorithms, or other technique depending on the application. For example, tube-shaped objects, may merit a Frangi vesselness filter or a Gabor filter. These filters may be used alone or in combination with one or more other filters to determine the candidate pixels/voxels. In certain embodiments, the preset model corresponds to a shape of the object to be detected. In some embodiments, the candidate pixel/voxels may then be processed using neural networks trained to classify the object within image data, and the object is identified within the image data. In some embodiments, the object identified may be localized by curve fitting or other techniques.

By using model-based filtering of image data to identify candidate pixels/voxels and processing only the candidate pixels/voxels by a neural network, systems and methods described herein may enhance the identification and/or classification of the object. The systems and methods described herein may also reduce the amount of time to identify an object, despite the added number of steps (e.g., applying a model then processing with a neural network rather than providing the data directly to the neural network).

An ultrasound imaging system according to an example of the present disclosure may include an ultrasound probe configured to acquire signals for generating an ultrasound image, and a processor configured to generate a first dataset comprising a first set of display data representative of the image from the signals, select a first subset of the first set of display data from the first dataset by applying a model to the first dataset, wherein the model is based on a property of an object to be identified in the image, select a second subset of data points from the first subset that represent the object, and generate a second set of display data from the second subset of data points, wherein the second set of display data is representative of the object within the image.

A method according to an example of the present disclosure may include processing a first dataset of an image with a model to generate a second dataset smaller than the first dataset, wherein the second dataset is a subset of the first dataset, and wherein the model is based, at least in part, on a property of an object to be identified in the image, analyzing the second dataset to identify which data points of the second dataset include the object, and outputting the data points of the second dataset identified as including the object as a third dataset, wherein the third dataset is output for display.

In accordance with an example of the present disclosure, a non-transitory computer-readable medium may contain instructions, that when executed, may cause an imaging system to process a first dataset of an image with a model, wherein the model is based on a property of an object to be identified in the image and based on the model, output a second dataset, wherein the second dataset is a subset of the first dataset, analyze the second dataset to determine which data points of the second dataset include the object and output a third dataset including the data points of the second dataset determined to include the object, and generate a display including the third dataset.

### BRIEF DESCRIPTION OF THE DRAWINGS

FIG. 1 illustrates an overview of the principles of the present disclosure.

FIG. 2 illustrates data processing steps for catheter identification in a 3D ultrasound volume according to principles of the present disclosure.

FIG. 3 is a block diagram of an ultrasound system in accordance with principles of the present disclosure.

FIG. 4 is a block diagram illustrating an example processor in accordance with principles of the present disclosure.

FIG. 5 is a block diagram of a process for training and deployment of a neural network in accordance with the principles of the present disclosure.

FIG. 6 is an illustration of a neural network in accordance with the principles of the present disclosure.

FIG. 7 is an illustration of a neural network in accordance with the principles of the present disclosure.

FIG. 8 is an illustration of a neural network in accordance with the principles of the present disclosure.

FIG. 9 illustrates a process of tri-planar extraction in accordance with the principles of the present disclosure.

FIG. 10 is an illustration of a neural network in accordance with principles of the present disclosure.

FIG. 11 shows example images of outputs of object identifiers in accordance with principles of the present disclosure.

FIG. 12 illustrates an example of a localization process for a catheter in accordance with principles of the present disclosure.

FIG. 13 shows example images of a catheter before and after localization in accordance with principles of the present disclosure.

FIG. 14 illustrates an overview of a method to identify an object in an image in accordance with principles of the present disclosure.

### DETAILED DESCRIPTION

The following description of certain embodiments is merely exemplary in nature and is in no way intended to limit the invention or its applications or uses. In the following detailed description of embodiments of the present systems and methods, reference is made to the accompanying drawings which form a part hereof, and which are shown by way of illustration specific embodiments in which the described systems and methods may be practiced. These embodiments are described in sufficient detail to enable those skilled in the art to practice presently disclosed systems and methods, and it is to be understood that other embodiments may be utilized and that structural and logical changes may be made without departing from the spirit and scope of the present system. Moreover, for the purpose of clarity, detailed descriptions of certain features will not be discussed when they would be apparent to those with skill in the art so as not to obscure the description of the present system. The following detailed description is therefore not to be taken in a limiting sense, and the scope of the present system is defined only by the appended claims.

Machine learning techniques, such as neural networks and deep learning algorithms, have provided advances in analyzing medical images, even lower resolution ones, which has improved the ability to identify and localize objects in images. These techniques may be used for diagnosis or for assessing a treatment (e.g., confirming placement of an implant). However, many machine learning techniques are still computationally complex and processing medical images, especially three-dimensional medical images, may require significant amounts of time. This may limit the practicality of using machine learning in real-time applications, such as interventional procedures.

As disclosed herein, images may be pre-processed by one or more techniques to select voxels of interest (VOI) prior to being analyzed by a neural network. Techniques for pre-processing may include, but not limited to, applying a filter, a first-stage neural network with less accuracy and/or complexity than the neural network, an algorithm, image segmentation, planar extraction from 3D patches, or combinations thereof. For simplicity, the pre-processing techniques may be referred to as a model and the model may be applied to an image. However, it is understood that the model may include multiple techniques. In some examples, the pre-processing may utilize prior knowledge of the object to be identified in the images (e.g., a known interventional device, an anatomical feature with relatively uniform appearance across subjects). Prior knowledge may include a property of the object, such as the shape, size or acoustic signal of the object. The pre-processing may reduce the amount of data that the neural network processes. Reducing the amount of data may reduce the time required for the neural network to identify an object in the image. The reduction in the time required by the neural network may be greater than the time required for pre-processing. Thus, the overall time to identify the object in the image may be reduced when compared to providing the images directly to the neural network.

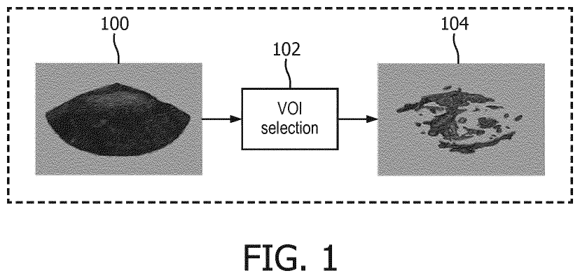

An overview of the principles of the present disclosure is provided in FIG. 1. An image 100 may be a 2D, 3D, or 4D image. In some examples, it may be a medical image, such as one acquired by an ultrasound imaging system, a computed tomography system, or a magnetic resonance imaging system. The image may be provided for pre-processing to select VOI as indicated by block 102 (in the case of a 2D image, pixels of interest (POI) would be selected). As mentioned previously, the pre-processing may utilize a model that may be based, at least in part, on a property of the object to be identified. For example, when a catheter is used during a cardiac intervention, the prior knowledge would be the tubular shape of the catheter. Continuing this example, a Frangi vesselness filter or a Gabor filter may be applied to the image 100 at block 102 to select the VOI. Other examples of objects include guide wires, cardiac plugs, artificial heart valves, valve clips, closure devices, and annuloplasty systems.

The model included in block 102 may output an image 104 that only includes the VOI. The VOI may include voxels that include the object to be identified as well as some false positive voxels from other areas and/or objects in the image 100. In the catheter example, the VOI may include voxels that include the catheter as well as some false positive voxels from the tissue or other elements. The image 104 may be provided to a neural network (not shown in FIG. 1) for further processing.

In some applications, allowing the pre-processing to include false positives may allow the pre-processing to take less time than if more precision were required. However, even with the false positives, the data included in image 104 may be significantly less than the data included in image 100. This may allow the neural network that receives the image 104 to provide results more quickly than if the neural network had received image 100. In some applications, the neural network may provide more accurate results based on image 104 rather than image 100.

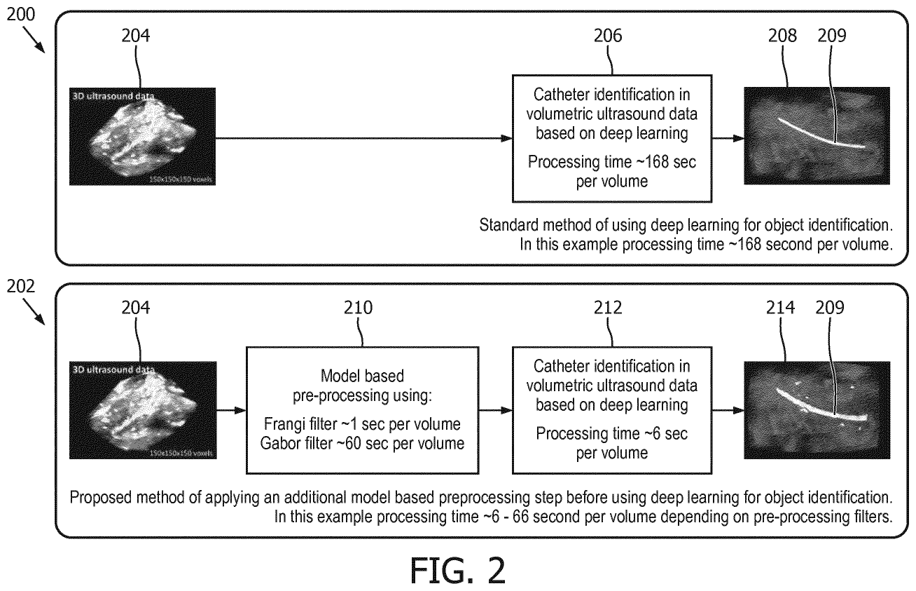

FIG. 2 illustrates data processing steps for catheter identification in an ultrasound volume according to principles of the present disclosure. Block 200 illustrates the situation when an ultrasound volume is provided directly to a neural network. Block 202 illustrations the situation when the ultrasound volume is provided for pre-processing prior to being provided to the neural network. Both blocks 200 and 202 have a 150×150×150 voxel ultrasound volume 204 of tissue with a catheter. In block 200, the ultrasound volume 204 is processed by deep learning algorithms (e.g., a neural network) at block 206 to generate an output volume 208 where the catheter 209 has been identified. The deep learning algorithm took approximately 168 seconds to process the 150×150×150 voxels by a deep learning framework on a standard nVidia graphical processing unit.

In block 202, the ultrasound volume 204 is provided first for pre-processing at block 210 to select VOI. If a Frangi filter is used, it takes approximately 1 second to process the 150×150×150 voxels. If a Gabor filter is used, it takes approximately 60 seconds to process the voxels. Both of these computation times are based on a standard central processing unit without code optimization. The Frangi and Gabor filters were use merely as illustrative examples. Other filters or techniques could be used for the pre-processing step in other examples. The VOI from the pre-processing at block 210 are provided for processing by deep learning algorithms at block 212. The deep learning algorithm generates an output volume 214 where the catheter 209 has been identified. The deep learning algorithm took approximately 6 seconds to process the 150×150×150 voxels. Thus, although block 202 includes an extra step compared to block 200, the process in block 202 only took 7-66 seconds compared to the 168 seconds of block 200.

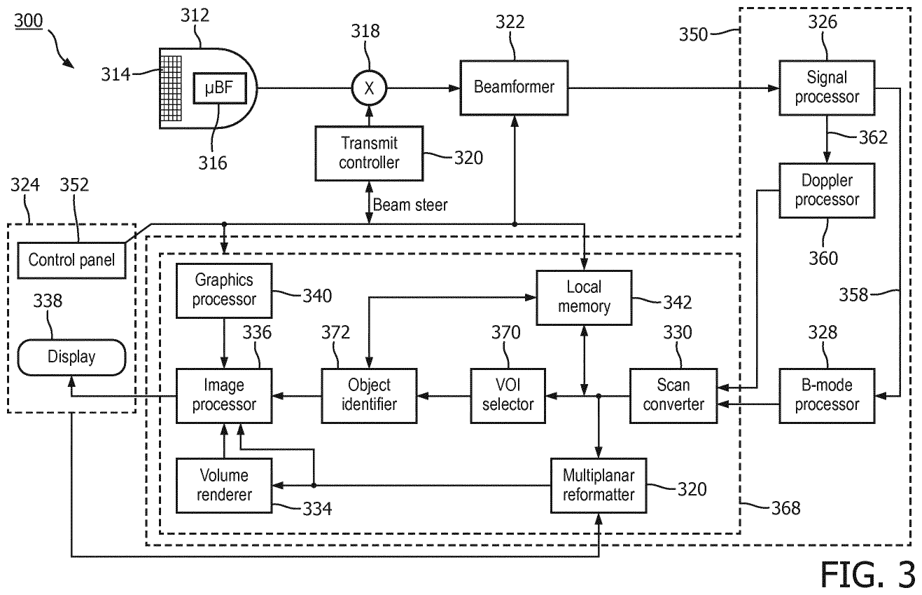

FIG. 3 shows a block diagram of an ultrasound imaging system 300 constructed in accordance with the principles of the present disclosure. An ultrasound imaging system 300 according to the present disclosure may include a transducer array 314, which may be included in an ultrasound probe 312, for example an external probe or an internal probe such as an Intra Cardiac Echography (ICE) probe or a Trans Esophagus Echography (TEE) probe. In other embodiments, the transducer array 314 may be in the form of a flexible array configured to be conformably applied to a surface of subject to be imaged (e.g., patient). The transducer array 314 is configured to transmit ultrasound signals (e.g., beams, waves) and receive echoes responsive to the ultrasound signals. A variety of transducer arrays may be used, e.g., linear arrays, curved arrays, or phased arrays. The transducer array 314, for example, can include a two dimensional array (as shown) of transducer elements capable of scanning in both elevation and azimuth dimensions for 2D and/or 3D imaging. As is generally known, the axial direction is the direction normal to the face of the array (in the case of a curved array the axial directions fan out), the azimuthal direction is defined generally by the longitudinal dimension of the array, and the elevation direction is transverse to the azimuthal direction.

In some embodiments, the transducer array 314 may be coupled to a microbeamformer 316, which may be located in the ultrasound probe 312, and which may control the transmission and reception of signals by the transducer elements in the array 314. In some embodiments, the microbeamformer 316 may control the transmission and reception of signals by active elements in the array 314 (e.g., an active subset of elements of the array that define the active aperture at any given time).

In some embodiments, the microbeamformer 316 may be coupled, e.g., by a probe cable or wirelessly, to a transmit/receive (T/R) switch 318, which switches between transmission and reception and protects the main beamformer 322 from high energy transmit signals. In some embodiments, for example in portable ultrasound systems, the T/R switch 318 and other elements in the system can be included in the ultrasound probe 312 rather than in the ultrasound system base, which may house the image processing electronics. An ultrasound system base typically includes software and hardware components including circuitry for signal processing and image data generation as well as executable instructions for providing a user interface.

The transmission of ultrasonic signals from the transducer array 314 under control of the microbeamformer 316 is directed by the transmit controller 320, which may be coupled to the T/R switch 318 and a main beamformer 322. The transmit controller 320 may control the direction in which beams are steered. Beams may be steered straight ahead from (orthogonal to) the transducer array 314, or at different angles for a wider field of view. The transmit controller 320 may also be coupled to a user interface 324 and receive input from the user's operation of a user control. The user interface 324 may include one or more input devices such as a control panel 352, which may include one or more mechanical controls (e.g., buttons, encoders, etc.), touch sensitive controls (e.g., a trackpad, a touchscreen, or the like), and/or other known input devices.

In some embodiments, the partially beamformed signals produced by the microbeamformer 316 may be coupled to a main beamformer 322 where partially beamformed signals from individual patches of transducer elements may be combined into a fully beamformed signal. In some embodiments, microbeamformer 316 is omitted, and the transducer array 314 is under the control of the beamformer 322 and beamformer 322 performs all beamforming of signals. In embodiments with and without the microbeamformer 316, the beamformed signals of beamformer 322 are coupled to processing circuitry 350, which may include one or more processors (e.g., a signal processor 326, a B-mode processor 328, a Doppler processor 360, and one or more image generation and processing components 368) configured to produce an ultrasound image from the beamformed signals (i.e., beamformed RF data).

The signal processor 326 may be configured to process the received beamformed RF data in various ways, such as bandpass filtering, decimation, I and Q component separation, and harmonic signal separation. The signal processor 326 may also perform additional signal enhancement such as speckle reduction, signal compounding, and noise elimination. The processed signals (also referred to as I and Q components or IQ signals) may be coupled to additional downstream signal processing circuits for image generation. The IQ signals may be coupled to a plurality of signal paths within the system, each of which may be associated with a specific arrangement of signal processing components suitable for generating different types of image data (e.g., B-mode image data, Doppler image data). For example, the system may include a B-mode signal path 358 which couples the signals from the signal processor 326 to a B-mode processor 328 for producing B-mode image data.

The B-mode processor can employ amplitude detection for the imaging of structures in the body. The signals produced by the B-mode processor 328 may be coupled to a scan converter 330 and/or a multiplanar reformatter 332. The scan converter 330 may be configured to arrange the echo signals from the spatial relationship in which they were received to a desired image format. For instance, the scan converter 330 may arrange the echo signal into a two dimensional (2D) sector-shaped format, or a pyramidal or otherwise shaped three dimensional (3D) format. The multiplanar reformatter 332 can convert echoes which are received from points in a common plane in a volumetric region of the body into an ultrasonic image (e.g., a B-mode image) of that plane, for example as described in U.S. Pat. No. 6,443,896 (Detmer). The scan converter 330 and multiplanar reformatter 332 may be implemented as one or more processors in some embodiments.

A volume renderer 334 may generate an image (also referred to as a projection, render, or rendering) of the 3D dataset as viewed from a given reference point, e.g., as described in U.S. Pat. No. 6,530,885 (Entrekin et al.). The volume renderer 334 may be implemented as one or more processors in some embodiments. The volume renderer 334 may generate a render, such as a positive render or a negative render, by any known or future known technique such as surface rendering and maximum intensity rendering.

In some embodiments, the system may include a Doppler signal path 362 which couples the output from the signal processor 326 to a Doppler processor 360. The Doppler processor 360 may be configured to estimate the Doppler shift and generate Doppler image data. The Doppler image data may include color data which is then overlaid with B-mode (i.e. grayscale) image data for display. The Doppler processor 360 may be configured to filter out unwanted signals (i.e., noise or clutter associated with non-moving tissue), for example using a wall filter. The Doppler processor 360 may be further configured to estimate velocity and power in accordance with known techniques. For example, the Doppler processor may include a Doppler estimator such as an auto-correlator, in which velocity (Doppler frequency) estimation is based on the argument of the lag-one autocorrelation function and Doppler power estimation is based on the magnitude of the lag-zero autocorrelation function. Motion can also be estimated by known phase-domain (for example, parametric frequency estimators such as MUSIC, ESPRIT, etc.) or time-domain (for example, cross-correlation) signal processing techniques. Other estimators related to the temporal or spatial distributions of velocity such as estimators of acceleration or temporal and/or spatial velocity derivatives can be used instead of or in addition to velocity estimators. In some embodiments, the velocity and power estimates may undergo further threshold detection to further reduce noise, as well as segmentation and post-processing such as filling and smoothing. The velocity and power estimates may then be mapped to a desired range of display colors in accordance with a color map. The color data, also referred to as Doppler image data, may then be coupled to the scan converter 330, where the Doppler image data may be converted to the desired image format and overlaid on the B-mode image of the tissue structure to form a color Doppler or a power Doppler image.

According to principles of the present disclosure, output from the scan converter 330, such as B-mode images and Doppler images, referred to collectively as ultrasound images, may be provided to a voxel of interest (VOI) selector 370. The VOI selector 370 may identify voxels of interest that may include an object to be identified in the ultrasound images. In some embodiments, the VOI selector 370 may be implemented by one or more processors and/or application specific integrated circuits. The VOI selector 370 may include one or more models which may each may include one or more filters, neural networks with less accuracy, algorithms, and/or image segmentors. In some embodiments, the VOI selector 370 may apply pre-existing knowledge of a property of the object (e.g., size, shape, acoustic properties) when selecting VOI. In some embodiments, the VOI selector 370 may include one or more preset models based on the object to be identified. In some embodiments, these preset models may be selected by a user via a user interface 324.

Optionally, in some embodiments, the VOI selector 370 may further reduce the data from the ultrasound images by converting 3D patches (e.g., cubes) of voxels into three orthogonal planes (e.g., tri-planar extraction). For example, the VOI selector 370 may take three orthogonal planes, each of which passes through the center of the patch. The remaining voxels in the patch may be discarded or ignored in some embodiments.

The VOI selected by the VOI selector 370 may be provided to an object identifier 372. The object identifier 372 may process the VOI received from the VOI selector 370 to identify which voxels of the VOI include the object of interest. For example, by classifying the voxels as including or not including the object of interest. In some embodiments, the object identifier 372 may output the original ultrasound image with the identified voxels highlighted (e.g., different color, different intensity). In other embodiments, the object identifier 372 may output the identified voxels to an image processor 336 for recombination with the original image.

Optionally, in some embodiments, the object identifier 372 and/or image processor 336 may further localize the object within the identified voxels generated by the object identifier 372. Localization may include curve fitting the identified voxels and/or other techniques based on knowledge of the object to be identified.

In some embodiments, the object identifier 372 may be implemented by one or more processors and/or application specific integrated circuits. In some embodiments, the object identifier 372 may include any one or more machine learning, artificial intelligence algorithms, and/or multiple neural networks. In some examples, object identifier 372 may include a deep neural network (DNN), a convolutional neural network (CNN), a recurrent neural network (RNN), an autoencoder neural network, or the like, to recognize the object. The neural network may be implemented in hardware (e.g., neurons are represented by physical components) and/or software (e.g., neurons and pathways implemented in a software application) components. The neural network implemented according to the present disclosure may use a variety of topologies and learning algorithms for training the neural network to produce the desired output. For example, a software-based neural network may be implemented using a processor (e.g., single or multi-core CPU, a single GPU or GPU cluster, or multiple processors arranged for parallel-processing) configured to execute instructions, which may be stored in computer readable medium, and which when executed cause the processor to perform a trained algorithm for identifying the object in the VOI received from the VOI selector 370.

In various embodiments, the neural network(s) may be trained using any of a variety of currently known or later developed learning techniques to obtain a neural network (e.g., a trained algorithm or hardware-based system of nodes) that is configured to analyze input data in the form of ultrasound images, measurements, and/or statistics and identify the object. In some embodiments, the neural network may be statically trained. That is, the neural network may be trained with a data set and deployed on the object identifier 372. In some embodiments, the neural network may be dynamically trained. In these embodiments, the neural network may be trained with an initial data set and deployed on the object identifier 372. However, the neural network may continue to train and be modified based on ultrasound images acquired by the system 300 after deployment of the neural network on the object identifier 372.

In some embodiments, the object identifier 372 may not include a neural network and may instead implement other image processing techniques for object identification such as image segmentation, histogram analysis, edge detection or other shape or object recognition techniques. In some embodiments, the object identifier 372 may implement a neural network in combination with other image processing methods to identify the object. The neural network and/or other elements included in the object identifier 372 may be based on pre-existing knowledge of the object of interest. In some embodiments, the neural network and/or other elements may be selected by a user via the user interface 324.

Output (e.g., B-mode images, Doppler images) from the object identifier 372, the scan converter 330, the multiplanar reformatter 332, and/or the volume renderer 334 may be coupled to an image processor 336 for further enhancement, buffering and temporary storage before being displayed on an image display 338. For example, in some embodiments, the image processor 336 may receive the output of the object identifier 372 that identifies the voxels including the object to be identified. The image processor 336 may overlay the identified voxels onto the original ultrasound image. In some embodiments, the voxels provided by the object identifier 372 may be overlaid in a different color (e.g., green, red, yellow) or intensity (e.g., maximum intensity) than the voxels of the original ultrasound image. In some embodiments, the image processor 336 may provide only the identified voxels provided by the object identifier 372 such that only the identified object is provided for display.

Although output from the scan converter 330 is shown as provided to the image processor 336 via the VOI selector 370 and object identifier 372, in some embodiments, the output of the scan converter 330 may be provided directly to the image processor 336. A graphics processor 340 may generate graphic overlays for display with the images. These graphic overlays can contain, e.g., standard identifying information such as patient name, date and time of the image, imaging parameters, and the like. For these purposes the graphics processor may be configured to receive input from the user interface 324, such as a typed patient name or other annotations. The user interface 344 can also be coupled to the multiplanar reformatter 332 for selection and control of a display of multiple multiplanar reformatted (MPR) images.

The system 300 may include local memory 342. Local memory 342 may be implemented as any suitable non-transitory computer readable medium (e.g., flash drive, disk drive). Local memory 342 may store data generated by the system 300 including ultrasound images, executable instructions, imaging parameters, training data sets, or any other information necessary for the operation of the system 300.

As mentioned previously system 300 includes user interface 324. User interface 324 may include display 338 and control panel 352. The display 338 may include a display device implemented using a variety of known display technologies, such as LCD, LED, OLED, or plasma display technology. In some embodiments, display 138 may comprise multiple displays. The control panel 352 may be configured to receive user inputs (e.g., exam type, preset model for object to be identified). The control panel 352 may include one or more hard controls (e.g., buttons, knobs, dials, encoders, mouse, trackball or others). In some embodiments, the control panel 352 may additionally or alternatively include soft controls (e.g., GUI control elements or simply, GUI controls) provided on a touch sensitive display. In some embodiments, display 338 may be a touch sensitive display that includes one or more soft controls of the control panel 352.

In some embodiments, various components shown in FIG. 3 may be combined. For instance, image processor 336 and graphics processor 340 may be implemented as a single processor. In another example, the VOI selector 370 and object identifier 372 may be implemented as a single processor. In some embodiments, various components shown in FIG. 3 may be implemented as separate components. For example, signal processor 326 may be implemented as separate signal processors for each imaging mode (e.g., B-mode, Doppler). In some embodiments, one or more of the various processors shown in FIG. 3 may be implemented by general purpose processors and/or microprocessors configured to perform the specified tasks. In some embodiments, one or more of the various processors may be implemented as application specific circuits. In some embodiments, one or more of the various processors (e.g., image processor 336) may be implemented with one or more graphical processing units (GPU).

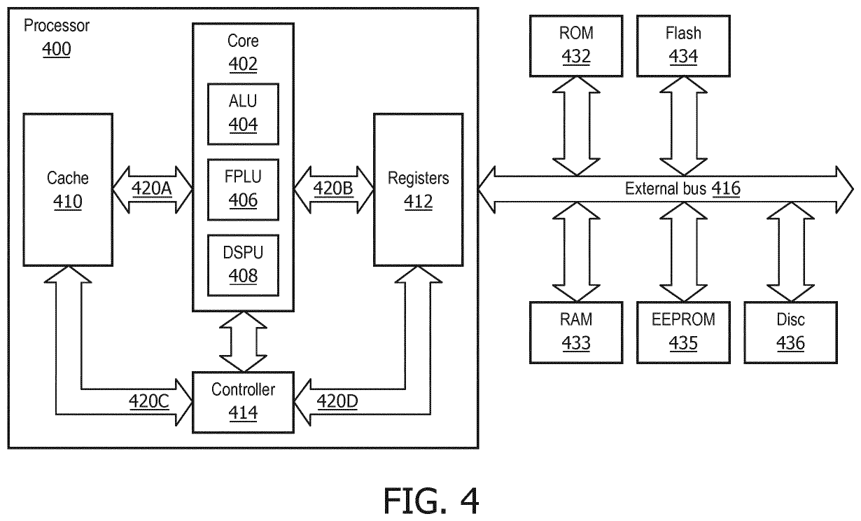

FIG. 4 is a block diagram illustrating an example processor 400 according to principles of the present disclosure. Processor 400 may be used to implement one or more processors and/or controllers described herein, for example, image processor 336 shown in FIG. 3 and/or any other processor or controller shown in FIG. 3. Processor 400 may be any suitable processor type including, but not limited to, a microprocessor, a microcontroller, a digital signal processor (DSP), a field programmable array (FPGA) where the FPGA has been programmed to form a processor, a graphical processing unit (GPU), an application specific circuit (ASIC) where the ASIC has been designed to form a processor, or a combination thereof.

The processor 400 may include one or more cores 202. The core 402 may include one or more arithmetic logic units (ALU) 404. In some embodiments, the core 402 may include a floating point logic unit (FPLU) 406 and/or a digital signal processing unit (DSPU) 408 in addition to or instead of the ALU 404.

The processor 400 may include one or more registers 412 communicatively coupled to the core 402. The registers 412 may be implemented using dedicated logic gate circuits (e.g., flip-flops) and/or any memory technology. In some embodiments the registers 412 may be implemented using static memory. The register may provide data, instructions and addresses to the core 402.

In some embodiments, processor 400 may include one or more levels of cache memory 410 communicatively coupled to the core 402. The cache memory 410 may provide computer-readable instructions to the core 402 for execution. The cache memory 410 may provide data for processing by the core 402. In some embodiments, the computer-readable instructions may have been provided to the cache memory 410 by a local memory, for example, local memory attached to the external bus 416. The cache memory 410 may be implemented with any suitable cache memory type, for example, metal-oxide semiconductor (MOS) memory such as static random access memory (SRAM), dynamic random access memory (DRAM), and/or any other suitable memory technology.

The processor 400 may include a controller 414, which may control input to the processor 400 from other processors and/or components included in a system (e.g., control panel 352 and scan converter 330 shown in FIG. 3) and/or outputs from the processor 400 to other processors and/or components included in the system (e.g., display 338 and volume renderer 334 shown in FIG. 3). Controller 414 may control the data paths in the ALU 404, FPLU 406 and/or DSPU 408. Controller 414 may be implemented as one or more state machines, data paths and/or dedicated control logic. The gates of controller 414 may be implemented as standalone gates, FPGA, ASIC or any other suitable technology.

The registers 412 and the cache 410 may communicate with controller 414 and core 402 via internal connections 420A, 420B, 420C and 420D. Internal connections may implemented as a bus, multiplexor, crossbar switch, and/or any other suitable connection technology.

Inputs and outputs for the processor 400 may be provided via a bus 416, which may include one or more conductive lines. The bus 416 may be communicatively coupled to one or more components of processor 400, for example the controller 414, cache 410, and/or register 412. The bus 416 may be coupled to one or more components of the system, such as display 338 and control panel 352 mentioned previously.

The bus 416 may be coupled to one or more external memories. The external memories may include Read Only Memory (ROM) 432. ROM 432 may be a masked ROM, Electronically Programmable Read Only Memory (EPROM) or any other suitable technology. The external memory may include Random Access Memory (RAM) 433. RAM 433 may be a static RAM, battery backed up static RAM, Dynamic RAM (DRAM) or any other suitable technology. The external memory may include Electrically Erasable Programmable Read Only Memory (EEPROM) 435. The external memory may include Flash memory 434. The external memory may include a magnetic storage device such as disc 436. In some embodiments, the external memories may be included in a system, such as ultrasound imaging system 300 shown in FIG. 3, for example local memory 342.

In some embodiments, the system 300 can be configured to implement a neural network included in the VOI selector 370 and/or object identifier 372, which may include a CNN, to identify an object (e.g., determine whether an object or a portion thereof is included in a pixel or voxel of an image). The neural network may be trained with imaging data such as image frames where one or more items of interest are labeled as present. Neural network may be trained to recognize target anatomical features associated with specific medical exams (e.g., different standard views of the heart for echocardiography) or a user may train neural network to locate one or more custom target anatomical features (e.g., implanted device, catheter).

In some embodiments, a neural network training algorithm associated with the neural network can be presented with thousands or even millions of training data sets in order to train the neural network to determine a confidence level for each measurement acquired from a particular ultrasound image. In various embodiments, the number of ultrasound images used to train the neural network(s) may range from about 50,000 or less to 200,000 or more. The number of images used to train the network(s) may be increased if higher numbers of different items of interest are to be identified, or to accommodate a greater variety of patient variation, e.g., weight, height, age, etc. The number of training images may differ for different items of interest or features thereof, and may depend on variability in the appearance of certain features. For example, tumors typically have a greater range of variability than normal anatomy. Training the network(s) to assess the presence of items of interest associated with features for which population-wide variability is high may necessitate a greater volume of training images.

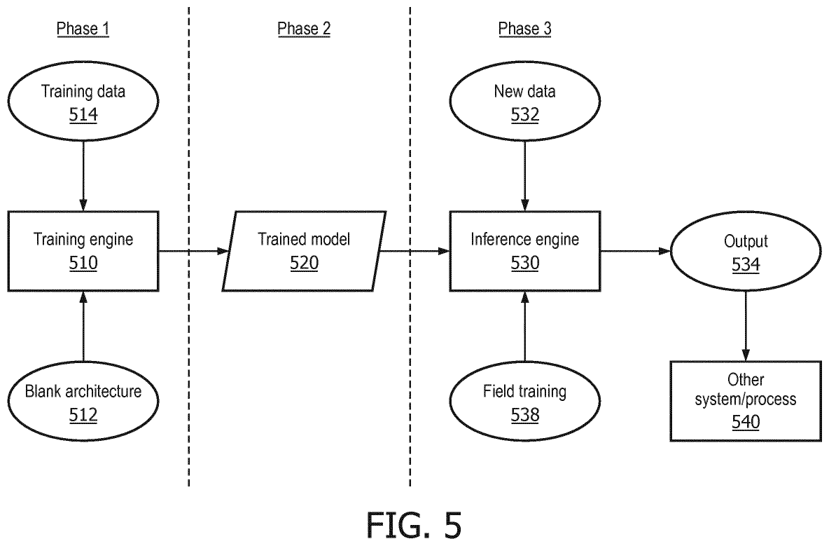

FIG. 5 shows a block diagram of a process for training and deployment of a neural network in accordance with the principles of the present disclosure. The process shown in FIG. 5 may be used to train a neural network included in the VOI selector 370 and/or object identifier 372. The left hand side of FIG. 5, phase 1, illustrates the training of a neural network. To train the neural network, training sets which include multiple instances of input arrays and output classifications may be presented to the training algorithm(s) of the neural network(s) (e.g., AlexNet training algorithm, as described by Krizhevsky, A., Sutskever, I. and Hinton, G. E. “ImageNet Classification with Deep Convolutional Neural Networks,” NIPS 2012 or its descendants). Training may involve the selection of a starting network architecture 512 and the preparation of training data 514. The starting network architecture 512 may be a blank architecture (e.g., an architecture with defined layers and arrangement of nodes but without any previously trained weights) or a partially trained network, such as the inception networks, which may then be further tailored for classification of ultrasound images. The starting architecture 512 (e.g., blank weights) and training data 514 are provided to a training engine 510 for training the model. Upon sufficient number of iterations (e.g., when the model performs consistently within an acceptable error), the model 520 is said to be trained and ready for deployment, which is illustrated in the middle of FIG. 5, phase 2. The right hand side of FIG. 5, or phase 3, the trained model 520 is applied (via inference engine 530) for analysis of new data 532, which is data that has not been presented to the model during the initial training (in phase 1). For example, the new data 532 may include unknown images such as live ultrasound images acquired during a scan of a patient (e.g., cardiac images during an echocardiography exam). The trained model 520 implemented via engine 530 is used to classify the unknown images in accordance with the training of the model 520 to provide an output 534 (e.g., voxels including the identified object). The output 534 may then be used by the system for subsequent processes 540 (e.g., output of a neural network of the VOI selector 370 may be used as input for the object identifier 372).

In the embodiments where the trained model 520 is used to implement a neural network of the object identifier 372, the starting architecture may be that of a convolutional neural network, or a deep convolutional neural network, which may be trained to perform image frame indexing, image segmentation, image comparison, or any combinations thereof. With the increasing volume of stored medical image data, the availability of high-quality clinical images is increasing, which may be leveraged to train a neural network to learn the probability of a given pixel or voxel includes an object to be identified (e.g., catheter, valve clip). The training data 514 may include multiple (hundreds, often thousands or even more) annotated/labeled images, also referred to as training images. It will be understood that the training image need not include a full image produced by an imagining system (e.g., representative of the full field of view of an ultrasound probe or entire MRI volume) but may include patches or portions of images of the labeled item of interest.

In various embodiments, the trained neural network may be implemented, at least in part, in a computer-readable medium comprising executable instructions executed by a processor, e.g., object identifier 372 and/or VOI selector 370.

For training with medical images, the class imbalance may be an issue. That is, there may be significantly more pixels or voxels without the object to be identified (e.g., tissue) than pixels or voxels including the object. For example, the ratio of catheter voxels vs. non-catheter voxels is commonly less than 1/1000. To compensate, a two-step training of the neural network(s) may be performed in some examples as described below.

First, the number of imbalanced voxels in training images may be re-sampled on non-catheter voxels to obtain the same amount as catheter voxels. These balanced samples train the neural networks. Then, the training images are validated on the trained models to select the falsely classified voxels, which are used to update the networks for finer optimization. Specifically, unlike when the neural network is deployed in the object identifier 372, the training process is applied in the whole ultrasound image rather than only VOI provided by the VOI selector 370. This update step reduces the class imbalance by dropping out the easiest sample points (so-called two stage training).

In some embodiments, the parameters of networks may be learned by minimizing the cross-entropy, using the Adam optimizer for faster convergence. During the two-step training, the cross-entropy is characterized into a different form to balance the class distribution. In the first training stage, the cross-entropy is characterized in a standard format. However, during the updating, the function is redefined as weighted cross-entropy. These different entropies avoid the bias in the updating stage, which occurs due to the number of false positives being usually 5 to 10 times larger than the positive training samples in the second stage. As a result of the weighted cross-entropy, the networks tend to preserve more object voxels (e.g., catheter) than discarding them after the classification. The weighted cross-entropy is formulated as:

Loss(y,p)=−(1−w)y log(p)−w(1−p) Equation 1

Where they indicates the label of the sample, while p is the class probability of the sample, and parameter w is the sample class ratio among the training samples.

During the training, in some embodiments, the dropout may be used to avoid overfitting with 50% probability in fully connect layers (FCs) of a convolutional network together with an L2 regularization with 10⁻⁵ strength. In some embodiments, the initial learning rate may be set to be 0.001 and resealed by a factor 0.2 after every 5 epochs. Meanwhile, to generalize the network in orientation and image intensity variation, data augmentation techniques like rotation, mirroring, contrast and brightness transformations may additionally be applied. In some embodiments, the mini-batch size may be 128, and the total training epoch may be 20 which are around 25 k in the first training, while iterations in the second training are around 100 k.

The above two-step training method is provided only as an example. Other multi-step or single-step training methods may be used in other examples.

Returning to the VOI selector 370, in some embodiments, the VOI selector 370 may include a filter such as a Gabor filter or a Frangi vesselness filter to select candidate voxels. In some cases, use of a filter may result in a large number of false-positives due to weak voxel discrimination, especially in noisy and/or low-quality 3D images. A large number of false positives may cause a larger than necessary data set to be provided to the object identifier 372 for analysis. This may reduce the speed of the object identifier 371.

In some embodiments, to reduce the number of false positives, the VOI selector 370 may optionally include an additional model. For example, a Frangi filter may be used in conjunction with an adaptive thresholding method. In this example, an image volume is first filtered by the Frangi filter with a pre-defined scale and resealed to a unit interval [0,1], V. After the Frangi filtering, an adaptive thresholding method may be applied to V to coarsely select N voxels with the highest vesselness response. Again, a Frangi filter is provided only as an example (e.g., for finding tubular structures). Other filters may also be used (e.g., based on prior knowledge of the shape or other characteristics of the object to be detected). The thresholding method may find the top N possible voxels in V. Because the filter response has a large variance in different images, the adaptive tuning of the threshold can gradually select N voxels by iteratively increasing or decreasing the threshold T based on the image itself. In some examples, the initial threshold may be set to T=0.3. The value of N may be selected to balance the efficiency of the VOI selector 370 and/or object identifier 372 classification and/or classification performance. In some applications, the value of N may range from 10 k to 190 k voxels with a step size of 10 k. In some examples, the values may be obtained by averaging of all testing volumes through three-fold cross validation. Pseudocode for the adaptive thresholding is shown below:

Require: filtered volume V, required voxel number N and initial threshold T

Apply threshold to V by the initial threshold value T. Find the remained voxels, which is larger than T, with amount of K.

if K < N then

while K < N do

T = T − 0.01.
Apply thresholding to V by T, find number of voxels K larger
than T.

end while

else if K > N then

while K > N do

T = T + 0.01.
Do thresholding on V by T, find number of voxels K larger than
T.

end while

end if
return The voxels with response larger than adapted threshold T.

In other examples, other techniques for reducing false positives may be used. For example, a fixed value thresholding method may be used. Furthermore, although the example above discusses the use of adaptive thresholding in combination with a filter, adaptive thresholding or other technique may be used in conjunction with a model and/or neural network in other embodiments.

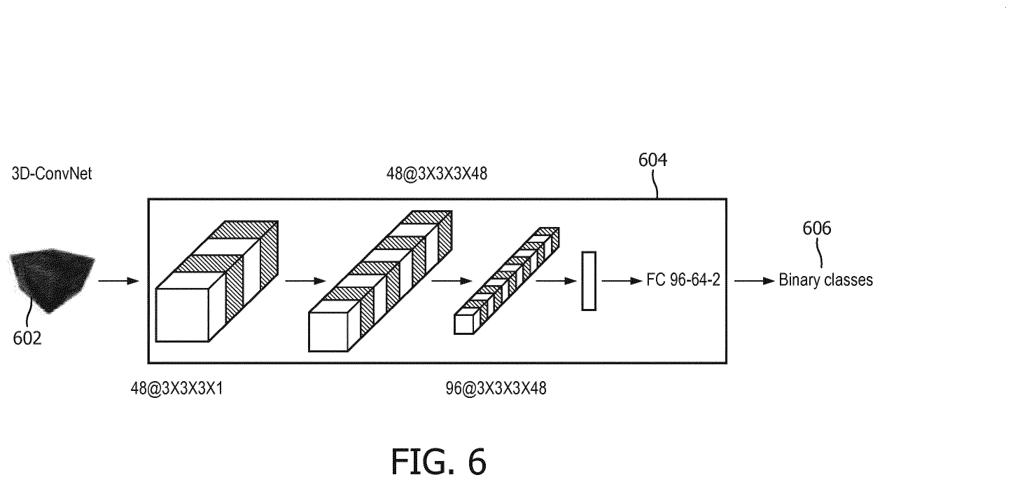

In some embodiments, the VOI output by the VOI selector 370 may be received and processed by the object identifier 372. For example, the object identifier 372 may include a 3D convolutional network that analyzes the VOI. In some embodiments, the VOI may be subdivided into 3D patches (e.g., cubes) and analyzed by the 3D convolutional network. For voxel-wise classification of volumetric data, in some embodiments, the object identifier 372 may process 3D local information by a neural network to classify the VOI provided by the VOI selector 370. In some embodiments, the neural network may be a convolutional neural network. In some embodiments, the classification may be a binary classification, such as containing or not containing the object of interest. In some embodiments, the voxels may be classified based on their 3D neighborhoods. For example, as shown in FIG. 6, for each candidate voxel located at the center of a 3D cube 602, the cube 602 may be processed by a 3D convolutional network 604 to output the classification 606 of the voxels. However, when using a 3D data cube as input, this approach includes many parameters in the neural network, which may hamper the efficiency of the voxel-wise classification in the image volume. In some examples, to preserve the 3D information and yet reduce the operations performed by the object identifier 372, 2D slices may be extracted from each cube (e.g., 3D patch), where each slice is taken from a different angle through the cube. In some embodiments, the multi-planar extraction may be performed by the VOI selector 370. In other embodiments, the multi-planar extraction may be performed by the object identifier 372.

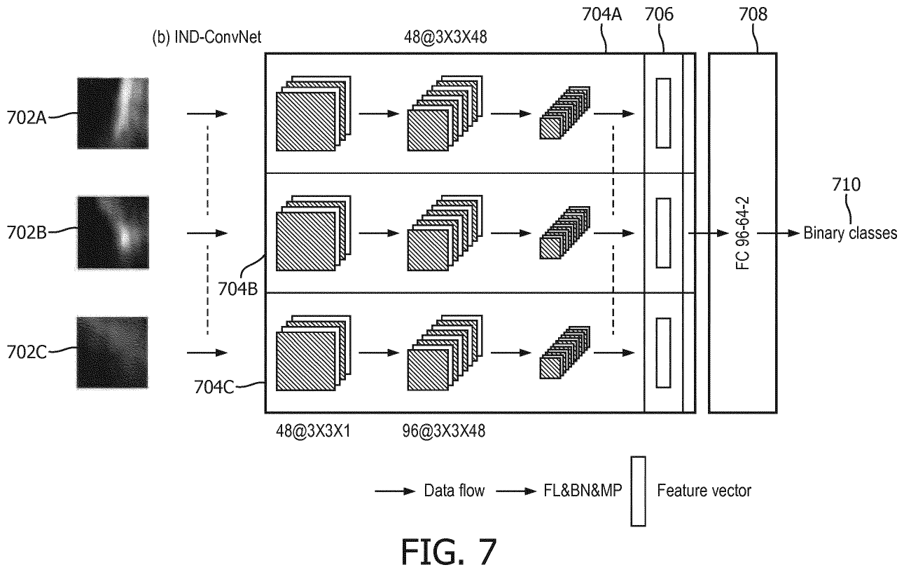

As shown in FIG. 7, in some embodiments, each extracted slice 702A-C may be provided to a separate respective neural network 704A-C. The extracted feature vectors 706 from the slices may be concatenated to feed them into fully connected layers (FCs) 708 to output the binary classes 710 of the voxels. Although this may preserve 3D information by a slicing approach, multiple individual neural network branches may lead to redundancy, which may provide sub-optimal application and computation time.

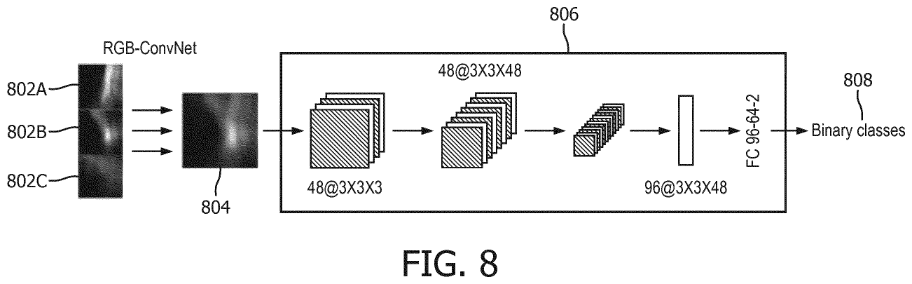

As shown in FIG. 8, in some embodiments, the extracted slices 802A-C may be reorganized into red-green-blue (RGB) channels 804. The RGB channels 804 are then provided to a single neural network 806 to output the binary classes 808. However, in some applications, this may cause the spatial information between each slice to be processed rigidly by convolutional filters at the first stage of the convolutional network of the neural network 806. With shallow processing, only low-level features may be processed and may not fully exploit the spatial relationship between the slices in some applications.

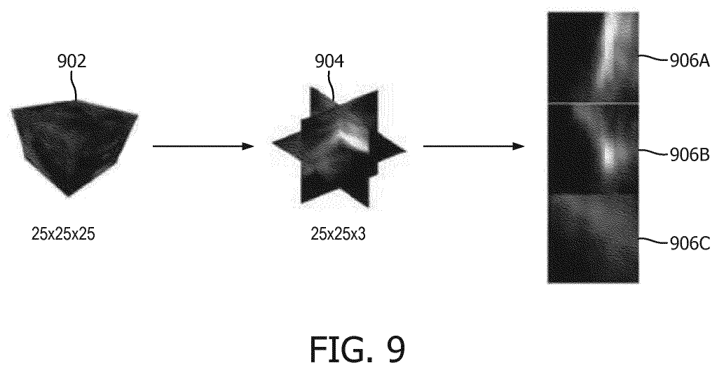

FIG. 9 illustrates a process of tri-planar extraction according to an embodiment of the disclosure. Based on the VOI provided by the VOI selector 370, a cube 902 may be obtained for each VOI, with the VOI located at the center of the cube. Then, three orthogonal planes passing through the center 904 of the cube 902 are extracted. The three orthogonal planes 906A-C are then provided as inputs to a neural network and/or other object identification technique of the object identifier 372. In some examples, the cube may be 25×25×25 voxels, which may be larger than a typical catheter diameter of 4-6 voxels. However, other sized cubes may be used in other examples based, at least in part, on a size of the object to be identified.

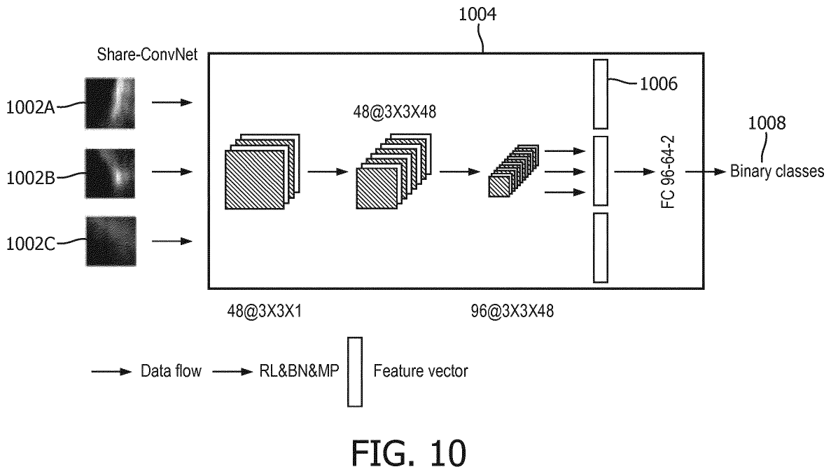

FIG. 10 shows a neural network according to an embodiment of the disclosure. Instead of training a neural network for each slice as shown in FIG. 7, a single neural network 1004, such as a convolutional network, may be trained to receive all three slices 1002A-C from tri-planar extraction as an input in some embodiments. All feature vectors 1006 from the shared convolutional network may be concatenated to form a longer feature vector for classification in some embodiments. The single neural network 1004 may output a binary classification 1008 of the voxels in the planes 1002A-C. In some applications, the neural network 1004 may exploit the spatial correlation of the slices 1002A-C in a high-level feature space.

The neural networks shown in FIGS. 6, 7, 9, and/or 10 may be trained as describe above with reference to FIG. 5 in some embodiments.

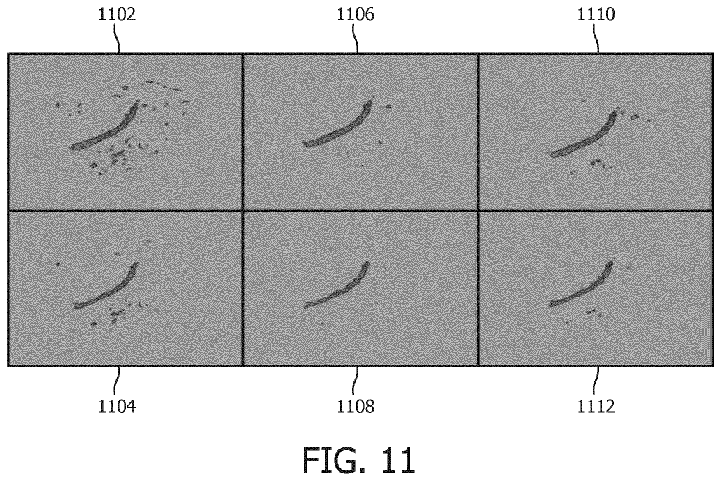

FIG. 11 shows example outputs of object identifiers according to various embodiments of the disclosure. All of the example outputs shown in FIG. 11 we generated from 3D ultrasound images (e.g., volumes) including a catheter. Panes 1102 and 1104 show the voxels output as including the catheter from an object identifier including a neural network as shown in FIG. 8. Pane 1102 was generated by the neural network from an original volume acquired by an ultrasound imaging system. Pane 1104 was generated by the neural network from the output of a VOI selector. Panes 1106 and 1108 show the voxels output as including the catheter from an object identifier including a neural network as shown in FIG. 10. Pane 1106 was generated by the neural network from an original volume acquired by an ultrasound imaging system. Pane 1108 was generated by the neural network from the output of a VOI selector. Panes 1110 and 1112 show the voxels output as including the catheter from an object identifier including a neural network as shown in FIG. 7. Pane 1110 was generated by the neural network from an original volume acquired by an ultrasound imaging system. Pane 1112 was generated by the neural network from the output of a VOI selector.

As shown in FIG. 11, all three neural networks provide outputs with less noise when generating outputs based on the output of the VOI selector. Thus, in some applications, not only does pre-processing the 3D image to select VOI increase the speed of the neural network, it may also improve the performance of the neural network. Furthermore, in some applications an object identifier including a neural network as shown in FIG. 10 may provide outputs with less noise object identifiers including neural networks as shown in FIG. 7 or 8.

In some applications, the voxels classified as including the object to be identified may include some outliers as can be seen in the “debris” surrounding the identified catheter in FIG. 11. This may be due, in some cases, to blurry tissue boundaries or catheter-like anatomical structures. Optionally, in some embodiments, after the voxels including the object to identified have been classified by the object identifier 372 (e.g., as classified by a neural network), the object may be further localized by additional techniques. These techniques may be performed by the object identifier 372 and/or the image processor 336 in some embodiments. In some embodiments, a pre-defined model and curve fitting techniques may be used.

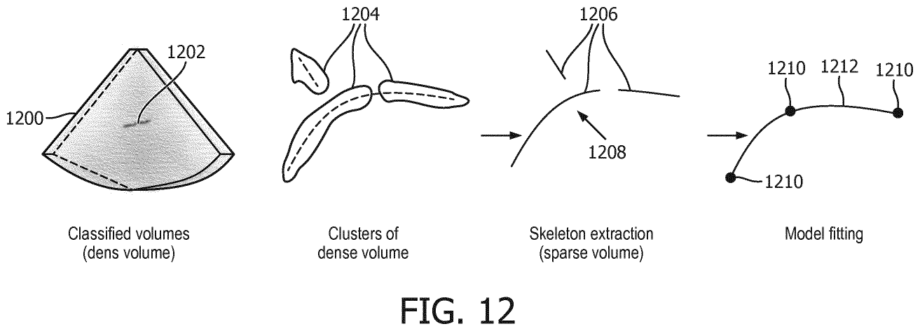

FIG. 12 illustrates an example of the localization process in the case of a catheter according to an embodiment of the disclosure. In this example, a curved cylinder model with a fixed radius may be used. The volume 1200 of voxels classified as including the catheter 1202 may be processed by connectivity analysis to generate clusters 1204. The cluster skeletons 1206 are extracted to generate a sparse volume 1208. A fitting stage is then performed. During fitting, multiple control points 1210 (e.g., three points as shown in FIG. 12) may be automatically and randomly selected from the sparse volume 1208 and ordered in orientation by principal component analysis. The reordered points 1210 may ensure cubic spline fitting passes the points in sequential order. This may generate the catheter-model skeleton 1212. In some embodiments, the localized skeleton 1212 with the highest number of inliers in the volume 1200 may be adopted as the fitted catheter. In some embodiments, the inliers may be determined by their Euclidean distances to the skeleton 1212.

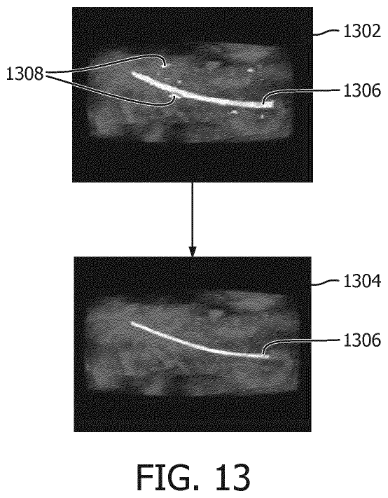

FIG. 13 shows example images of a catheter before and after localization according to an embodiment of the disclosure. Pane 1302 shows a 3D ultrasound image with voxels classified as a catheter 1306 highlighted. Outliers in the tissue are also highlighted as being identified as part of the catheter 1306. Pane 1304 shows a 3D ultrasound image with voxels classified the catheter 1306 after a localization process (e.g., the process described in reference to FIG. 12) has been performed. As shown in FIG. 13, the voxels including the catheter 1306 have been more narrowly defined and the outliers 1308 have been eliminated. As shown in FIG. 13, performing a localization process on the output of a neural network and/or other classification scheme of the object identifier may improve visualization of the identified object in some applications.

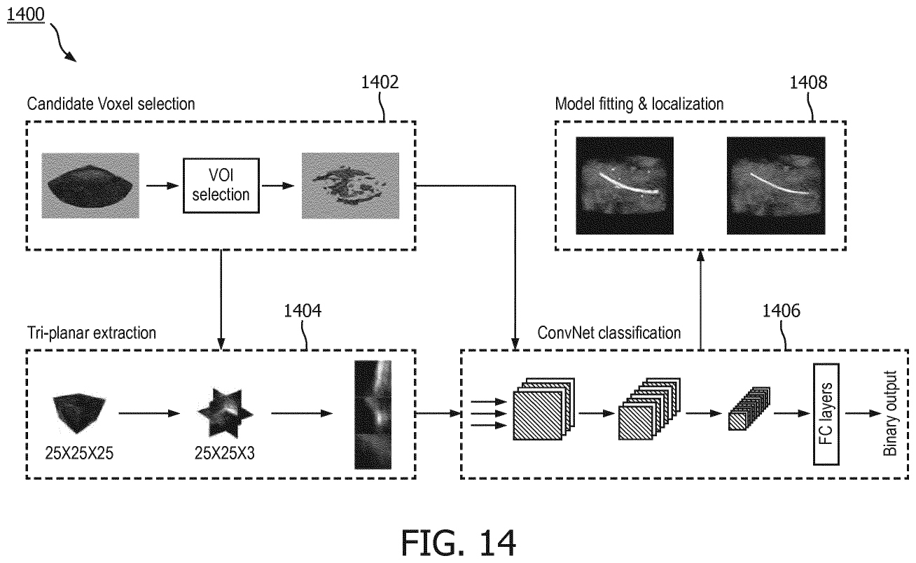

FIG. 14 illustrates an overview of a method 1400 to identify an object in an image according to an embodiment of the disclosure. At block 1402, an image or image volume (e.g., a 3D ultrasound image) is pre-processed by a model to select data of interest from display data representing the image or image volume. In some embodiments, the model may be implemented by a processor, which may be referred to as a VOI selector, such as VOI selector 370. The data of interest may contain, or have a possibility of containing, an object to be identified. The data of interest output by the preprocessing may be a subset of the display data (e.g., a second dataset).

Optionally, at block 1404, when the second dataset is a 3D dataset (e.g., a volume), the second dataset may be subdivided into 3D patches (e.g., cubes). Multiple planes (e.g., slices) may then be extracted from each 3D patch. For example, in some embodiments, three orthogonal planes passing through the center of each 3D patch may be extracted. In some embodiments, the planar extraction may be performed by the VOI selector. In other embodiments, the planar extraction may be performed by an object identifier, such as object identifier 372. In some embodiments, the object identifier may be implemented by a processor. In some embodiments, a single processor may implement both the VOI selector and the object identifier. A set of planes may then be output by the VOI selector or object identifier.

At block 1406, the second dataset may be processed to identify data points (e.g., voxels or pixels) in the second dataset that include the object to be identified. For example, the data points may be analyzed to determine whether or not they include the object. In some embodiments, the data points of a 3D dataset may be processed by a neural network, for example, the neural network shown in FIG. 6. In some embodiments, the processing may be performed by the object identifier, which may include the neural network. In other embodiments, the data points of a 2D dataset may be processed by a neural network similar to the one shown in FIG. 6, but the neural network may have been trained on 2D image data sets. In some embodiments, the data points of the second dataset identified as including the object of interest may be output as a third dataset, which may be a subset of the second dataset. The third dataset may represent the object. In some embodiments, the third dataset may be used to generate display data to output to display and/or recombined with the original image or image volume for display.

In other embodiments, at block 1406, the planes extracted from the 3D patches at block 1404 may be processed to identify the data points in the planes including the object to be identified. In some embodiments, the data points may be processed by a neural network, for example, the neural network shown in FIGS. 7, 8, and/or 10. In some embodiments, the processing may be performed by the object identifier, which may include the neural network. In some embodiments, the data points of the planes identified as including the object of interest may be output as a third dataset, which may be a subset of the data points included in the planes. In some embodiments, the third dataset may be output for display and/or recombined with the original image volume for display.

Optionally, in some embodiments, the object may be further localized in the third dataset at block 1408. In some embodiments, a localization process may be performed by the object identifier or an image processor, such as image processor 336. In some embodiments, localization may include applying a model and/or curve fitting techniques to the third dataset based, at least in part, on knowledge of the object to be identified in the volume (e.g., a property of the object). In some embodiments, the localized voxels and/or pixels may be output as a fourth dataset, which may be a subset of the third dataset. In some embodiments, the fourth dataset may be output for display and/or recombined with the original image or image volume for display.

Prior to the method 1400 illustrated in FIG. 14, in some embodiments, one or more neural network for selecting the data points and/or identifying the data points including an object to be identified may be trained by one or more methods described previously herein.

As disclosed herein, images may be pre-processed by one or more techniques to select voxels of interest (VOI) prior to being analyzed by a neural network. The pre-processing may reduce the amount of data that the neural network processes. Optionally, the data may be further reduced by extracting orthogonal planes from the set of VOI and providing the orthogonal planes to the neural network. Reducing the amount of data may reduce the time required for the neural network to identify an object in the image. The reduction in the time required by the neural network may be greater than the time required for pre-processing. Thus, the overall time to identify the object in the image may be reduced when compared to providing the images directly to the neural network. Optionally, the object identified by the neural network may be further localized by curve-fitting or other techniques. This may enhance the visualization of the object provided by the neural network in some applications.

Although the examples described herein discuss processing of ultrasound image data, it is understood that the principles of the present disclosure are not limited to ultrasound and may be applied to image data from other modalities such as magnetic resonance imaging and computed tomography.

In various embodiments where components, systems and/or methods are implemented using a programmable device, such as a computer-based system or programmable logic, it should be appreciated that the above-described systems and methods can be implemented using any of various known or later developed programming languages, such as “C”, “C++”, “C #”, “Java”, “Python”, and the like. Accordingly, various storage media, such as magnetic computer disks, optical disks, electronic memories and the like, can be prepared that can contain information that can direct a device, such as a computer, to implement the above-described systems and/or methods. Once an appropriate device has access to the information and programs contained on the storage media, the storage media can provide the information and programs to the device, thus enabling the device to perform functions of the systems and/or methods described herein. For example, if a computer disk containing appropriate materials, such as a source file, an object file, an executable file or the like, were provided to a computer, the computer could receive the information, appropriately configure itself and perform the functions of the various systems and methods outlined in the diagrams and flowcharts above to implement the various functions. That is, the computer could receive various portions of information from the disk relating to different elements of the above-described systems and/or methods, implement the individual systems and/or methods and coordinate the functions of the individual systems and/or methods described above.

In view of this disclosure it is noted that the various methods and devices described herein can be implemented in hardware, software and firmware. Further, the various methods and parameters are included by way of example only and not in any limiting sense. In view of this disclosure, those of ordinary skill in the art can implement the present teachings in determining their own techniques and needed equipment to affect these techniques, while remaining within the scope of the invention. The functionality of one or more of the processors described herein may be incorporated into a fewer number or a single processing unit (e.g., a CPU) and may be implemented using application specific integrated circuits (ASICs) or general purpose processing circuits which are programmed responsive to executable instruction to perform the functions described herein.

Although the present system may have been described with particular reference to an ultrasound imaging system, it is also envisioned that the present system can be extended to other medical imaging systems where one or more images are obtained in a systematic manner. Accordingly, the present system may be used to obtain and/or record image information related to, but not limited to renal, testicular, breast, ovarian, uterine, thyroid, hepatic, lung, musculoskeletal, splenic, cardiac, arterial and vascular systems, as well as other imaging applications related to ultrasound-guided interventions. Further, the present system may also include one or more programs which may be used with conventional imaging systems so that they may provide features and advantages of the present system. Certain additional advantages and features of this disclosure may be apparent to those skilled in the art upon studying the disclosure, or may be experienced by persons employing the novel system and method of the present disclosure. Another advantage of the present systems and method may be that conventional medical image systems can be easily upgraded to incorporate the features and advantages of the present systems, devices, and methods.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

1. An ultrasound imaging system comprising:
an ultrasound probe configured to acquire signals for generating an ultrasound image; and
a processor configured to:
generate a first dataset comprising a first set of display data representative of the image from the signals;
select a first subset of the first set of display data from the first dataset by applying a model to the first dataset, wherein the model is based on a property of an object to be identified in the image;
select a second subset of data points from the first subset that represent the object; and
generate a second set of display data from the second subset of data points, wherein the second set of display data is representative of the object within the image.

2. The ultrasound imaging system of claim 1, wherein the processor is further configured to:
subdivide first subset into cubes;
extract multiple planes from each cube; and
select the second subset of data points only from data points of the first subset included in the multiple planes.

3. The ultrasound imaging system of claim 2, wherein the multiple planes include three orthogonal planes, each of which pass through the center of the cube.

4. The ultrasound imaging system of claim 1, wherein the processor includes a neural network.

5. The ultrasound imaging system of claim 4, wherein the neural network is trained by a two-step training process.

6. The ultrasound imaging system of claim 1, wherein the model includes at least one of a Frangi vesselness filter or a Gabor filter.

7. The ultrasound imaging system of claim 6, wherein the model further includes an adaptive thresholding algorithm.

8. The ultrasound imaging system of claim 1, wherein the processor is further configured to select a third subset from the second subset by applying at least one curve-fitting technique to the data points of the second subset, wherein the third subset represents a localization of the object.

9. The ultrasound imaging system of claim 1, further comprising a user interface configured to receive a user input that selects one of a plurality of preset models as the model.

10. A method of identifying an object in an image, the method comprising:
processing a first dataset of an image with a model to generate a second dataset smaller than the first dataset, wherein the second dataset is a subset of the first dataset, and wherein the model is based, at least in part, on a property of an object to be identified in the image;
analyzing the second dataset to identify which data points of the second dataset include the object; and
outputting the data points of the second dataset identified as including the object as a third dataset, wherein the third dataset is output for display.

11. The method of claim 10, further comprising receiving a user input including a type of object to be identified.

12. The method of claim 10, wherein analyzing the second dataset includes providing the second dataset to a neural network.

13. The method of claim 10, further comprising:
subdividing the second dataset into 3D patches;
extracting at least one slice from each 3D patch; and
outputting data points included in the at least one slice as the second dataset for analyzing.

14. The method of claim 10, wherein the property of the object includes at least one of a size, a shape, or an acoustic signal.

15. The method of claim 10, further comprising localizing the object in the third dataset using at least one curve-fitting techniques and outputting a fourth dataset including the object.

16. The method of claim 15, wherein localizing the object includes cubic spline fitting.

17. A non-transitory computer readable medium including instructions that when executed cause an imaging system to:
process a first dataset of an image with a model, wherein the model is based on a property of an object to be identified in the image and based on the model, output a second dataset, wherein the second dataset is a subset of the first dataset;
analyze the second dataset to determine which data points of the second dataset include the object and output a third dataset including the data points of the second dataset determined to include the object; and
generate a display including the third dataset.

18. The non-transitory computer readable medium of claim 17, further including instructions that when executed cause the imaging system to:
perform tri-planar extraction on the second dataset, wherein only the data points extracted by the tri-planar extraction are output as the second dataset to be analyzed.

19. The non-transitory computer readable medium of claim 17, further including instructions that when executed cause the imaging system to localize the object within the third dataset.

20. The non-transitory computer readable medium of claim 17, wherein the model includes at least one of a Frangi vesseness filter or a Gabor filter.
